(19) United States
(12) Patent Application Publication
(10) Pub. No.: US 2004/0068190 A1
(43) Pub. Date: Apr. 8, 2004

(54) Imaging catheter with indicia and methods of use

(76) Inventor: Eduardo Ignacio Cespedes, Folsom, CA (US)

Correspondence Address: Nicola A. Pisano, Luce, Forward, Hamilton & Scripps LLP, 11988 El Camino Real, San Diego, CA, 92130, US

(21) Appl. No.: 10/265,515

(22) Filed: Oct. 4, 2002

(51) Int. Cl. A61B 8/14

(52) U.S. Cl. 600/466

(57) ABSTRACT

Apparatus and methods are provided for measuring axial displacement of an imaging catheter, and for using real-time images acquired by the imaging catheter to measure axial lengths of anatomical sites. The apparatus of the present invention includes a catheter having an imaging element disposed on a distal portion and indicia disposed on a proximal portion. The length of the catheter permits the indicia to be disposed at least partially external to a patient and visible to a medical practitioner when the imaging element is disposed within the blood vessel. Accordingly, the medical practitioner can observe the indicia and associate axial locations of the catheter indicated by the indicia with images obtained by the imaging element.

### FIELD OF THE INVENTION

The present invention relates to apparatus and methods for measuring axial displacement of an imaging catheter, and for using real-time images acquired by the imaging catheter to measure axial lengths of anatomical sites.

### BACKGROUND OF THE INVENTION

A large number of medical diagnostic and therapeutic procedures involve the percutaneous introduction of minimally invasive imaging instrumentation into a blood vessel. Exemplary imaging instrumentation includes Intravascular Ultrasound (“IVUS”) catheters, Optical Coherence Tomography (“OCT”) catheters, and Magnetic Resonance Imaging (“MRI”) catheters.

An often-used technique to determine axial displacement of an imaging catheter within the blood vessel is to couple the imaging catheter to a pullback system. The pullback system has one or more motors that are engaged to and may retract the imaging catheter at a controlled rate. The pullback system also may be electrically coupled to a data acquisition system that records axial displacement of the catheter during pullback. The axial displacement recorded by the data acquisition system may be correlated with displacement observed in imaging data acquired by the imaging catheter.

Pullback systems add cost to diagnostic or therapeutic procedures, increase the complexity of the procedures, and, because the catheter is mechanically coupled to the pullback system, reduce the clinician's degree of tactile sensation in maneuvering the catheter.

In view of these drawbacks, it would be desirable to provide inexpensive apparatus for measuring axial displacement of an imaging catheter within a blood vessel.

It also would be desirable to provide easy to use apparatus and methods for measuring axial displacement of an imaging catheter within a blood vessel.

It further would be desirable to provide apparatus and methods for measuring axial lengths of anatomical sites within a blood vessel.

It still further would be desirable to provide apparatus and methods to determine and retain the axial location of a particular anatomical site within a blood vessel, thereby facilitating relocation of an instrument to that anatomical site.

### SUMMARY OF THE INVENTION

In view of the foregoing, it is an object of the present invention to provide inexpensive apparatus for measuring axial displacement of an imaging catheter within a blood vessel.

It also is an object of the present invention to provide easy to use apparatus and methods for measuring axial displacement of an imaging catheter within a blood vessel.

It further is an object of the present invention to provide apparatus and methods for measuring axial lengths of anatomical sites within a blood vessel.

It still further is an object of the present invention to provide apparatus and methods to determine and retain the axial location of a particular anatomical site within a blood vessel, thereby facilitating relocation of an instrument to that anatomical site.

These and other objects of the present invention are accomplished by providing apparatus and methods for measuring axial displacement of an imaging catheter, and for using real-time images acquired by the imaging catheter to measure axial lengths of anatomical sites. The apparatus of the present invention includes a catheter having an imaging element disposed on a distal portion and indicia disposed on a proximal portion. The length of the catheter permits the indicia to be disposed at least partially external to a patient and visible to a medical practitioner when the imaging element is disposed within the blood vessel. Accordingly, the medical practitioner can observe the indicia and associate axial locations of the catheter indicated by the indicia with images obtained by the imaging element.

The indicia may embody various styles, including enumerated, width-coded, and color-coded bands.

The catheter also may include optional radiopaque markers disposed on the distal portion.

Pursuant to another aspect of the present invention, a clip may be provided to facilitate relocation of the imaging element to a particular axial location within the blood vessel. The clip is configured to releasably engage the catheter on the proximal portion and demarcate an index of the indicia that corresponds to the particular axial location.

Methods of using the apparatus of the present invention also are provided.

### BRIEF DESCRIPTIONS OF THE INVENTION

Further features of the present invention, its nature and various advantages will be more apparent from the accompanying drawings and the following detailed description of the preferred embodiments, in which:

FIG. 1 is a schematic perspective view of an imaging catheter of the present invention;

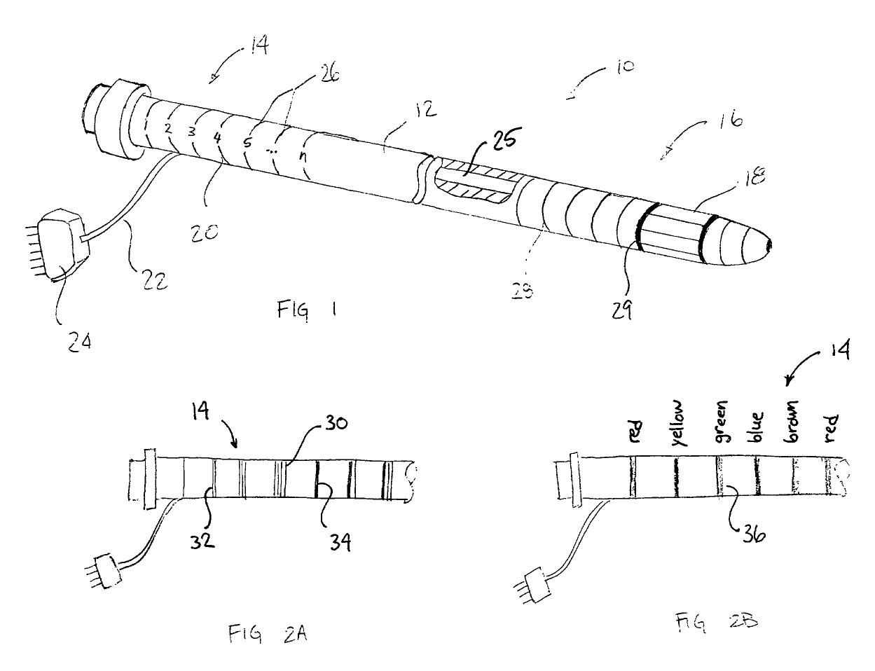

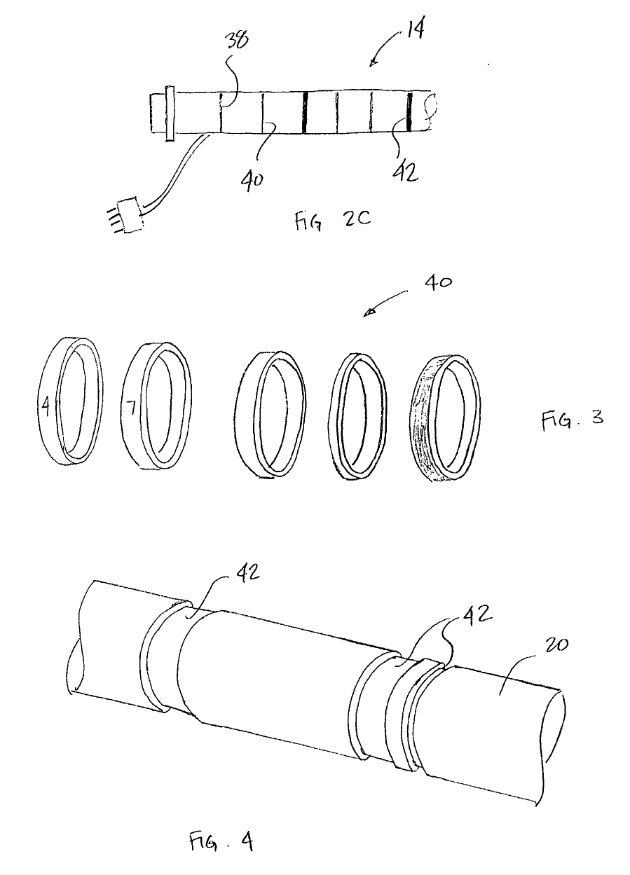

FIGS. 2A-2C are schematic partial side views of alternative styles of indicia disposed on the imaging catheter of the present invention;

FIG. 3 is a schematic perspective view of bands that may be used as the indicia of FIGS. 1 and 2A-C;

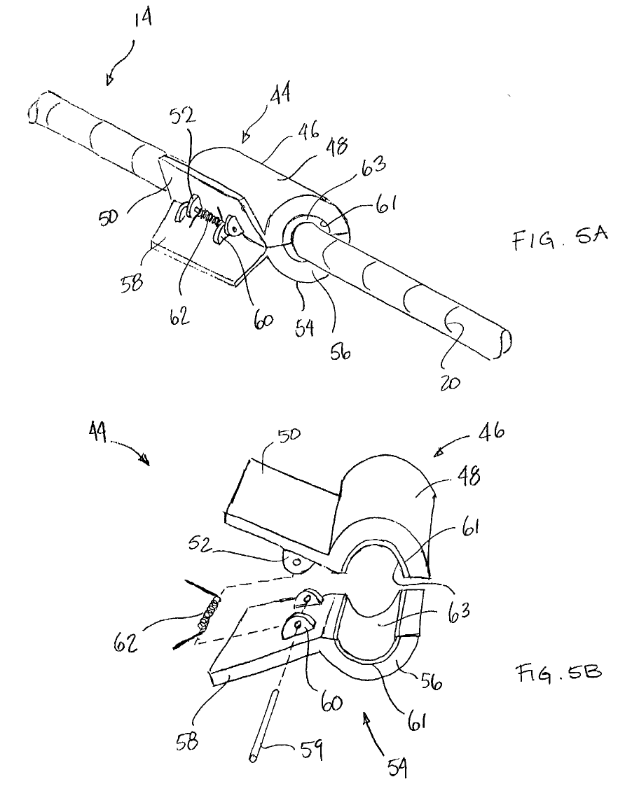

FIG. 4 is a schematic partial perspective view of a proximal portion of the imaging catheter of the present invention with recesses configured to accept the bands of FIG. 3;

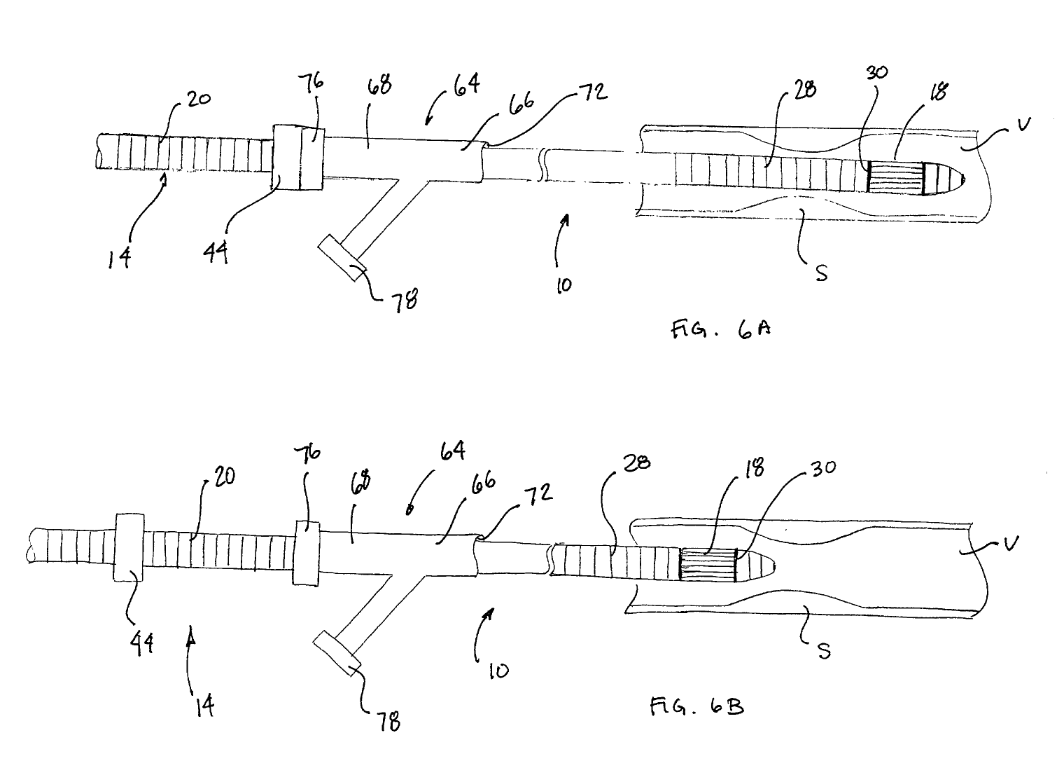

FIGS. 5A and 5B are, respectively, a schematic perspective view and an exploded perspective view of a clip for releasably engaging the imaging catheter of the present invention; and

FIGS. 6A and 6B are schematic side views describing a method of using the imaging catheter of the present invention.

### DETAILED DESCRIPTION OF THE INVENTION

The following description emphasizes inclusion of indicia on a proximal end of a phased-array intravascular ultrasound (IVUS) catheter. However, it is contemplated that the indicia may be used with catheters of any imaging modality, such as ultrasound, phased-array intravascular ultrasound, linear-array ultrasound, rotational ultrasound, forward-looking ultrasound, radially-looking ultrasound, magnetic resonance imaging, angiography, optical coherence tomography, and combinations thereof.

Referring to FIG. 1, a preferred embodiment of the present invention is described. Catheter 10 includes catheter body 12 having proximal portion 14, distal portion 16, imaging element 18 disposed on distal portion 16, and indicia 20 disposed on proximal portion 14. Imaging element 18 illustratively incorporates a phased-array IVUS transducer, which uses an array of discrete ultrasound elements that each provide image data. The image data from each element is combined to form a circumferential image of an interior of a blood vessel into which catheter 10 may be inserted. Phased-array IVUS systems are marketed by JOMED Inc., of Rancho Cordova, Calif., and are described, for example, in U.S. Pat. No. 6,283,920 to Eberle et al., which is incorporated herein by reference.

Catheter 10 also may include multiplexing circuitry, amplifiers, etc., per se known, which may be disposed on and/or electrically coupled to catheter 10. Imaging element 18 of catheter 10 is electrically coupled via cable 22 and connector 24 to an imaging system (not shown), per se known, that provides excitation waveforms to the imaging element, and interprets and displays data received therefrom. Catheter 10 optionally may incorporate at least one lumen 25 for irrigation, aspiration, or advancement of additional diagnostic or therapeutic instruments into the patient's blood vessel.

In the preferred embodiment of FIG. 1, indicia 20 incorporates enumerated bands 26 that encircle part or all of the circumference of catheter body 12. The distance between each successive band 26 preferably is on the scale of centimeters, but will depend on the application for which catheter 10 is used.

It will be apparent to one of ordinary skill in the art that, while enumerated bands 26 are numbered from “1” to “n” in FIG. 1, other ranges may be provided. For example, the number of each enumerated band 26 may correspond to the distance of that band from a reference feature, such as the distal or proximal end of catheter 10 or imaging element 18.

Pursuant to another aspect of the present invention, catheter 10 optionally may include radiopaque markers 28 disposed on distal portion 16 of catheter 10. Preferably, the distance between successive radiopaque markers 28 is equivalent to the distance between successive bands 26 of indicia 20. Alternatively, catheter 10 may be provided with radiopaque markers 28 in which the distance between adjacent radiopaque markers are larger or smaller than that between adjacent bands 26. In that case, a scaling factor may be provided or determined prior to insertion of catheter 10 within a patient.

As shown in FIG. 1, radiopaque markers 29 having a width greater than that of markers 28 may be provided immediately proximal and/or distal to imaging element 18 to respectively demarcate the proximal and/or distal extremities thereof.

Alternative styles of indicia 20 are schematically illustrated in FIGS. 2A-C. In FIG. 2A, indicia 20 includes width-coded bands 30, disposed on proximal portion 14 of catheter body 12 in a configuration analogous to Roman numerals. More specifically, each thin band 32 corresponds to, e.g., one unit length and each thick band 34 corresponds to, e.g., five unit lengths. Accordingly, to indicate, e.g., seven unit lengths, a combination of one thick band 34 and two thin bands 32 are provided.

In FIG. 2B, indicia 20 includes color-coded bands 36. Each successive band 36 may comprise a different color representing a different numerical value, or colors may be replicated for every, e.g., five bands in succession. In the latter embodiment, the distance between succeeding bands of the same color would correspond to, e.g., five unit lengths.

In FIG. 2C, indicia 20 also incorporates bands 38 of varying widths. Like width-coded bands 30 of FIG. 2A, bands 38 also include thin bands 40 and thick bands 42. The difference between the two embodiments lies in the configuration of the thick and thin bands along proximal portion 14. Specifically, in FIG. 2C, bands 38 are serially arranged so that thick bands 42 are interposed between a predetermined number of thin bands 40. Thus, each band 38 represents, e.g., one unit length from adjacent bands 38.

of course, it will be evident to one of ordinary skill in the art that catheter 10 may be provided with a combination of the styles of indicia 20 described hereinabove or with alternative styles. Furthermore, it will be evident that the distance between successive bands depends on the application for which catheter 10 is used. For example, for catheters to be used in treatment of abdominal aortic aneurysm, the distance between adjacent bands is preferably approximately 0.5-1.0 cm. For the treatment of vascular stenosis, the distance between adjacent bands may be on the order of millimeters. As will be apparent, a ruler-type scale, per se known, may be provided having indicia and sub-indicia (not shown).

During manufacture, indicia 20 may be printed or laser etched onto catheter 10. Alternatively, indicia 20 may comprise independently manufactured bands 40, as shown in FIG. 3, having numerals thereon, different widths, and/or a variety of colors. These bands may be embedded into catheter body 12 during manufacture of catheter body 12.

Alternatively, the bands may be made of a thermally responsive polymer that, upon exposure to heat, contracts bands 40 in diameter. Accordingly, during manufacture, bands 40 may be threaded onto proximal portion 14 of catheter body 12 and placed at predetermined locations along the axial length thereof. To ensure that bands 40 do not increase the outer diametrical profile of catheter 10 and to facilitate proper location of bands 40 on catheter body 12, catheter body 12 optionally may have recesses 42 (see FIG. 4) of widths approximately equal to those of corresponding bands 40 and depths approximately equal to the thickness of bands 40. Upon exposure to heat, bands 40 shrink in diameter to fixedly engage catheter body 12 and recesses 42, if present. It will be apparent to one of ordinary skill in the art that the widths and depths of recesses 42 should account for the slight change in the widths and thickness of bands 40 after bands 40 are exposed to heat.

Referring now to FIGS. 5A and 5B, imaging catheter 10 of the present invention further includes clip 44 that may be removably engaged to proximal portion 14 to demarcate a particular index of indicia 20. Clip 44 has first piece 46, which includes first jaw 48, first actuator lever 50, and first mounts 52 fixedly disposed on first actuator lever 50. Clip 44 also has second piece 54, which includes second jaw 56, second actuator lever 58, and second mounts 60 fixedly disposed on second actuator lever 58. First and second pieces 46 and 54 are rotatably coupled via shaft 59 that is disposed through first and second mounts 52 and 60. First and second jaws 48 and 56 comprise inner surfaces 61 that are contoured to engage proximal portion 14. Clip 44 optionally may incorporate lining 63 that is disposed on inner surfaces 61, and that is made of a material, e.g., rubber, that would resist longitudinal displacement of jaws 48 and 56 along proximal portion 14 of catheter 10, thus decreasing the likelihood that clip 44, and thus a particular index of indicia 20 demarcated by clip 44, will be inadvertently dislodged.

To maintain clip 44 in engagement with proximal portion 14, spring 62 preferably is provided to apply a spring force to actuator levers 50 and 58 that clamps jaws 48 and 56 together. The spring force can be overcome by squeezing actuator levers 50 and 58 together. Clip 44 is preferably made of sterilizable materials.

Referring now to FIGS. 6A and 6B, a method of using catheter 10 is provided. As shown in FIG. 6A, catheter 10 of the present invention is disposed through guide catheter 64. Guide catheter 64 includes body 66 having proximal Y-adaptor 68 and lumen 72. Y-adaptor 68 includes port 76 that accepts imaging catheter 10 of the present invention, and port 78 that may be used for irrigation, aspiration, and/or advancement of additional diagnostic or therapeutic instruments.

Upon insertion and placement of guide catheter 64 in a desired position within blood vessel V, imaging catheter 10 is advanced past port 76, through lumen 72, and out of guide catheter 64 to a target region within blood vessel V. As imaging element 18 is advanced past an area of interest, such as stenosis S, images acquired by the imaging element are displayed on a graphical user interface (not shown). Once imaging element 18 is advanced distal to stenosis S, as confirmed by observation of the images displayed on the graphical user interface, clip 44 may be engaged to proximal portion 14 of catheter 10 to demarcate the appropriate index of indicia 20 that corresponds to the distal position of stenosis S. Specifically, clip 44 is engaged to proximal portion 14 immediately proximal to port 76 of guide catheter 64, as shown in FIG. 6A.

Imaging catheter 10 then is proximally retracted across stenosis S. Once images displayed on the graphical user interface indicate that the proximal end of stenosis S has been reached, a clinician can note the appropriate index of indicia 20 that corresponds to the proximal end of stenosis S (i.e., the index of indicia 20 immediately proximal to port 76 of guide catheter 64). Subtraction of that index from the index demarcated by the distal edge of clip 44 affixed to proximal portion 14 equals the axial length of stenosis S. It will be evident to one of ordinary skill that the axial length of the target region, e.g. stenosis S, alternatively may be determined by counting the number of indices between the index demarcated by clip 44 and the index corresponding to the proximal end of the target region, for example, when catheter 10 is provided with indicia of the style described in FIGS. 2B-C. In this manner, a measurement of the axial length of an anatomical site within a patient's blood vessel may be obtained based on observation of images acquired by imaging element 18.

A further advantage of the present invention is that, after clip 44 is affixed to proximal portion 14 to demarcate a particular anatomical site of interest, such as the distal extremity of stenosis S, that site of interest can be immediately relocated if imaging element 18 is inadvertently or deliberately displaced therefrom. For example, if the medical practitioner displaces imaging element 18 during advancement of additional diagnostic or therapeutic instruments through lumen 25 of catheter 10 (see FIG. 1) or through port 78 of guide catheter 64, stenosis S can be easily and quickly relocated by distally advancing imaging catheter 10 until clip 44 contacts port 76, as in FIG. 6A. This disposes imaging element 18 just distal to stenosis S.

It will be apparent to one of ordinary skill that clip 44 may be used to demarcate and facilitate relocation of any anatomical sites of interest within blood vessel V. The above description emphasizes relocation of a stenosis only as an exemplary illustration of the use and advantages of the present invention.

While preferred illustrative embodiments of the present invention are described above, it will be apparent to one skilled in the art that various changes and modifications may be made therein without departing from the invention. The appended claims are intended to cover all such changes and modifications that fall within the true spirit and scope of the invention.

What is claimed is:

1. Apparatus for insertion into a blood vessel of a patient, comprising:
a catheter having a proximal portion, a distal portion, and a distal end;
an imaging element disposed on the distal portion; and
indicia disposed on the proximal portion, the indicia enabling visual confirmation of axial displacement of the imaging element within the blood vessel.

2. The apparatus of claim 1, further comprising at least one radiopaque marker disposed on the distal portion.

3. The apparatus of claim 1, further comprising a clip removably engaged to the proximal portion.

4. The apparatus of claim 1, wherein the indicia comprises a plurality of enumerated bands.

5. The apparatus of claim 4, wherein each of the plurality of enumerated bands corresponds to the distance of each of the plurality of enumerated bands from a reference feature.

6. The apparatus of claim 1, wherein the indicia comprises a plurality of width-coded bands.

7. The apparatus of claim 6, wherein the plurality of width-coded bands comprises a plurality of thick bands and a plurality of thin bands.

8. The apparatus of claim 1, wherein the indicia comprises a plurality of color-coded bands.

9. The apparatus of claim 8, wherein the plurality of color-coded bands comprises a predetermined number of different colors.

10. The apparatus of claim 1, wherein the imaging element is chosen from the group consisting of ultrasound transducers, phased-array intravascular ultrasound transducers, linear array ultrasound transducers, rotational ultrasound transducers, forward-looking ultrasound transducers, radially-looking ultrasound transducers, magnetic resonance imaging elements, optical coherence tomographers, and combinations thereof.

11. A kit for demarcating a particular axial location of an anatomical site within a blood vessel, the kit comprising:
a catheter having a proximal portion, a distal portion, and indicia disposed on the proximal portion, the indicia enabling visual confirmation of axial displacement of the catheter within the blood vessel; and
a clip configured to be removably engaged to the proximal portion to demarcate one index of the indicia.

12. The kit of claim 11, wherein the catheter further comprises an imaging element disposed on the distal portion.

13. The kit of claim 11, wherein the catheter further comprises a plurality of radiopaque markers disposed on the distal portion.

14. The kit of claim 11, wherein the clip further comprises a lining that resists axial dislodgement of the clip from the proximal portion.

15. The kit of claim 11, wherein the indicia comprises a plurality of enumerated bands.

16. The kit of claim 11, wherein the indicia comprises a plurality of width-coded bands.

17. The kit of claim 11, wherein the indicia comprises a plurality of color-coded bands.

18. A method of measuring axial length of an anatomical site within a blood vessel, the method comprising:
providing apparatus having a catheter comprising a distal portion, a proximal portion, an imaging element disposed on the distal portion, and indicia disposed on the proximal portion;
inserting the catheter into the blood vessel until images acquired by the imaging element indicate that the imaging element is disposed at a distal extremity of the anatomical site;
noting a first index of the indicia corresponding to the distal extremity;
proximally retracting the catheter while observing the images acquired by the imaging element;
ceasing retraction of the catheter when the images acquired by the imaging element indicate that the imaging element is disposed at a proximal extremity of the anatomical site;
noting a second index of the indicia corresponding to the proximal extremity; and
determining the axial length of the anatomical site based on the first index and the second index.

19. The method of claim 18, wherein providing apparatus comprises providing apparatus further comprising a clip, and wherein noting a first index comprises removably engaging the clip to the proximal portion of the catheter at the first index.

20. The method of claim 18, further comprising relocating the imaging element to the distal extremity of the anatomical site using the first index for guidance.

21. The method of claim 18, wherein determining the axial length comprises subtracting the first index from the second index.

22. The method of claim 18, wherein determining the axial length comprises counting the indicia disposed between the first index and the second index.
